(19) United States
(12) Patent Application Publication
(10) Pub. No.: US 2020/0383933 A1
(43) Pub. Date: Dec. 10, 2020

(54) METHOD FOR OBTAINING NANOSTRUCTURES WITH CAROTENOIDS AND NANOSTRUCTURES OBTAINED

(71) Applicants: UNIVERSIDAD DE CHILE, Santiago (CL); UNIVERSIDAD AUSTRAL DE CHILE, Valdivia (CL)

(72) Inventors: Felipe Andrés OYARZÚN AMPUERO, Santiago (CL); Javier MORALES VALENZUELA, Santiago (CL); Matías SILVA SILVA, Santiago (CL); Mariela INOSTROZA RIQUELME, Santiago (CL); Andrew QUEST, Santiago (CL); Marcelo KOGAN BOCIAN, Santiago (CL); Simón GUERRERO RIVERA, Santiago (CL); Ignacio MORENO VILLOSLADA, Valdivia (CL); Carlos Alberto ALARCÓN ALARCÓN, Valdivia (CL); Mario FLORES FLORES, Valdivia (CL); César Antonio TORRES GALLEGOS, Valdivia (CL)

(21) Appl. No.: 16/644,840

(22) PCT Filed: Sep. 5, 2017
(86) PCT No.: PCT/CL2017/050046

§ 371 Date: Mar. 5, 2020

(51) Int. Cl. A61K 9/51; A61K 9/107; A61K 31/122; C09B 61/00

(52) U.S. Cl. CPC A61K 9/5192; A61K 9/1075; B82Y 30/00; C09B 61/00; A61K 31/122

(57) ABSTRACT

The present invention relates to a method for obtaining nanostructures, specifically nanoemulsions and nanocapsules of carotenoids such as curcumin and astaxanthin. This method—using particular concentrations of the ingredients forming the nanostructures—allows high association efficiencies of the active ingredient in the nanostructures containing these ingredients and protection of these nanostructures from environmental factors such as oxidation and light. The invention also relates to the nanoemulsions and nanocapsules of curcumin and astaxanthin for use in the food, pharmaceutical and cosmetics industries, among others.

### TECHNICAL FIELD

The present invention relates to the technical field of nano-encapsulation of active compounds. Particularly, it addresses a method to obtain nanostructures, specifically nanoemulsions and nanocapsules of carotenoids such as curcumin and astaxanthin. The present invention also refers to curcumin and astaxanthin nanoemulsions and nanocapsules, to be used in the food, pharmaceutical, cosmetology industries or others.

### BACKGROUND TO THE INVENTION

Carotenoids are organic pigments that are found in abundance in nature. More than 600 of these compounds have been reported. Carotenoids are widely used in industry as dyes, but recently the therapeutic potential of some of these—such as curcumin and astaxanthin—has been discovered.

According to their structure most carotenoids are tetraterpinoids (C40), which correspond to 8 isoprenoid units, linked in such a way that the molecules are linear and symmetrical with two terminal rings. Due to their structure, carotenoids are hydrophobic, lipophilic molecules, insoluble in water and soluble in solvents such as acetone, alcohol and chloroform. They are molecules also characterized by being photosensitive and unstable to changes in pH and oxygen (Natália Mezzomo and Sandra R. S. Ferreira, “Carotenoids Functionality, Sources, and Processing by Supercritical Technology: A Review,” Journal of Chemistry, vol. 2016, 16 page, 2016).

Due to the physicochemical characteristics of these molecules, there has been a constant interest in searching their encapsulation by means of emulsions, particles with charged polymers and a mixture of both methods to increase their solubility in water. For example, the patent application WO 2009/093812 A2, proposes a method of co-polymerization of monomers to form a polymer, having these a hydrophobic group, to encapsulate carotenoids in general, mentioning astaxanthin among them. This patent does not mention the photoprotective effect of this polymer to environmental changes, either photolysis or changes in pH. At the same time, the patent application WO 2009/016091 A1 develops a method to encapsulating fat-soluble dyes and flavors—carotenoids among them—producing capsules of polymer-coated nanoemulsions. For this process they use high temperatures and a sucrose ester as emulsifier, so it requires a lot of energy to generate them. In none of the applications mentioned before the protection of the carotenoids or other liposoluble molecules against changes in the environment (e.g. light, oxidation, etc.) is ensured, nor is the charging efficiency of these molecules in their products evaluated.

In the case of curcumin and astaxanthin—since they are molecules of high commercial interest due to their therapeutic potential (as antioxidant, anti-inflammatory, antibacterial and anticarcinogenic, among others)—greater interest is generated. The main problem with these two molecules is that they are very poor candidates for traditional vehicularization and administration in water-containing media. For example, curcumin has a low solubility in aqueous media and is highly unstable (rapid hydrolysis due to changes in pH and oxygenation). So, it is therefore necessary to generate nanostructures that increase its solubility in water and protect them from these changes in the environment.

Curcumin and astaxanthin are molecules belonging to the carotenoid family having great therapeutic potential (antioxidant, anti-inflammatory, antibacterial and anti-cancer, among others). However, due to the physical-chemical characteristics of these molecules—among which a very low water solubility and very high environmental instability (light, oxygen and neutral pH, among other conditions) can be highlighted-their therapeutic potential is highly limited. In the case of postulating a product that considers the oral administration of these molecules and in an aqueous medium (drinks, tonics, juices, yogurt, soups, among others) the limitations of solubility, photolysis and oxidation become critical.

In the same vein, there is technical background on attempts to generate nanocapsules for these molecules through mixtures with cationic polymers or nanoemulsions developed with lipid compounds. For example, with respect to curcumin, they generated nanoemulsions with MCT-60 using Tween-80 and a serum protein concentrate WPC-70 [(Sari, T. P., Mann, B., Kumar, R., Singh, R. R. B., Sharma, R., Bhardwaj, M., & Athira, S. (2015). Preparation and characterization of nanoemulsion encapsulating curcumin. Food Hydrocolloids, 43, 540-546)). These nano-emulsions have a z-potential of maximum −6 mV, i.e., they form an unstable solution, and the protective effect of the nano-emulsion on curcumin with respect to light and oxidation was not evaluated. In addition, using a similar methodology in a previous work [(Abbas, Shabbar & Eric, Karangwa & Bashari, Mohanad & Hayat, Khizar & Hong, Xiao & Sharif, Hafiz & Zhang, Xiaoming (2014). Fabrication of polymeric nanocapsules from curcumin-loaded nanoemulsion templates by self-assembly. Ultrasonics Sonochemistry. 23)], the same kind of emulsions coated with starch modified with Octenyl succinic anhydride (OSA) were generated and then used as a cationic polymer coating, chitosan. In this work, neither the efficiency of the process nor whether these nanocapsules protect the curcumin from light and oxidation was evaluated.

On the other hand, curcumin emulsions were generated in corn oil produced by high pressure and high temperature homogenization for 10 minutes at 100° C. for its later use in alginate or carrageenan hydrogels [(Zhang, Z., Zhang, R., Zou, L., Chen, L., Ahmed, Y., Al Bishri, W. & McClements, D. J. (2016). Encapsulation of curcumin in polysaccharide-based hydrogel beads: Impact of bead type on lipid digestion and curcumin bioaccessibility. Food Hydrocolloids, 58, 160-170)] This method is therefore very energy-intensive and expensive for large-scale production.

U.S. Pat. No. 9,504,754 B2, and subsequent article by the same inventors [(Kumar, S., Kesharwani, S. S., Mathur, H., Tyagi, M., Bhat, G. J., & Tummala, H. (2016). Molecular complexation of curcumin with pH sensitive cationic copolymer enhances the aqueous solubility, stability and bioavailability of curcumin. European Journal of Pharmaceutical Sciences, 82, 86-96)] show an example where a cationic complexation of curcumin-polymer (Eudragit® E PO) was produced in a ratio of 1:2. So particles with a curcumin loading efficiency of 55.6% with respect to this polymer were generated. According to this background, this was the highest efficiency achieved, corresponding to a loss of curcumin of 44.4% in relation to the initial one, which is a very inefficient method with a high loss of active compound. Moreover, in this work they do not evaluate if these particles generate photoprotection of curcumin, explaining that, for this reason, all the tests in this work were performed in darkness.

With respect to astaxanthin, nanoemulsions of this molecule were generated with a non-ionic surfactant and palm olein as oil, by the HPH high pressure homogenization method [(Affandi, M. M., Julianto, T., & Majeed, A. (2011). Development and stability evaluation of astaxanthin nanoemulsion. Asian J Pharm Clin Res, 4(1), 142-148)]. Not knowing the protective effect of the emulsion, all procedures were performed in darkness.

On the other hand, astaxanthin nanocapsules were generated using lecithin and chitosan by aggregation and sonication [(Liu, N., Zhang, X., & Zhou, D. (2013). Preparation and properties research of astaxanthin loaded nanocapsules. Journal of Agricultural Science and Technology (Beijing), 15(6), 35-39)]. The encapsulation efficiency of astaxanthin was 51.02%, with a loading capacity of only 10.34% showing that this methodology is not optimal.

Consequently, there are no appropriate methods for carotenoid nanoencapsulation that are efficient in effectively loading the active ingredient, using low energy and associated costs, and that are optimal in aqueous media without loss of material. Also, there are no methods that—having a high loading efficiency—are able to protect these molecules from environmental changes caused by the effect of light, oxidation, changes in pH, etc. Therefore, it is necessary to generate an effective method to generate nanostructures, having the least loss of active ingredient, allowing it to be solubilized to an appropriate concentration in an aqueous environment and protecting the molecules contained in the nanostructures from environmental changes in order to generate a stable product over time and with uncountable opportunities in the industry.

### SUMMARY OF THE INVENTION

The present invention provides a highly efficient method for obtaining nanostructures with carotenoids as regards the charge of these molecules on the nanostructures obtained, which in turn provides appropriate protection to them against environmental factors such as light and oxidation.

To this end, the method for obtaining carotenoid nanostructures of the present invention includes the stages of mixing a carotenoid compound with an anionic surfactant, with an organic solvent that is soluble in water, and with a liquid oil, in particular mass proportions of 1:5-70:10-1000:30-250, respectively. Water is added to the abovementioned mixture in a ratio of 1:1-100, respectively, and the organic solvent is removed, thus obtaining a nanoemulsion.

The method of the invention includes optionally adding to the mixture of the carotenoid compound with an anionic surfactant, the water-miscible organic solvent, and the liquid oil, a second water-miscible organic solvent in a mass ratio of 1:10-20.

The method of the invention further includes adding a cationic polymer to the water of the corresponding step to form a cationic polymer solution and then either removing the solvents, or adding a cationic polymer solution to the obtained nanoemulsion to obtain in this way a nanoemulsion coated as a cationic nanocapsule. This cationic polymer solution is found at a concentration of 0.01-2% w/v in the final mixture.

Optionally the method of the invention allows obtaining anionic nanocapsules by mixing cationic nanocapsules with an anionic polymer solution in a concentration between 0.01-2% w/v in the final mixture.

In a preferred mode the nanostructures with carotenoids contain curcumin or astaxanthin.

If a formulation with curcumin is desired, the method includes the following steps:
(a) mixing curcumin with an anionic extract of lecithin, with ethanol, and with a liquid oil, in a mass ratio of 1:8, 6:114:34, respectively
(b) adding to the above mixture acetone in a ratio of 1:14;
(c) add to the above mixture water in a ratio of 1:36; and
(d) removing ethanol and acetone to obtain a nanoemulsion.

In the preferred mode wanting to obtain a cationic nanocapsule with curcumin, a cationic polymer is added to the water of the corresponding step to form a cationic polymer solution, and then the solvent removal step is performed, or alternatively a cationic polymer solution is added to the nano-emulsion obtained. In one preferred mode the cationic polymer is a cationic polymethacrylate and it is at a concentration between 0.01 and 1% w/v and in another preferred mode the cationic polymer is chitosan and it is at a concentration between 0.01 and 1% w/v.

The method of the invention also allows anionic nanocapsules from cationic nanocapsules with curcumin coated with cationic polymethacrylate where these are mixed with a solution of iota carrageenan in a concentration of 0.0765% w/v in a ratio of 1:1.

In another preferred mode of the invention the method allows to obtain astaxanthin nanostructures, where the method includes the following steps:
(a) mixing astaxanthin with an anionic extract of lecithin, with ethanol, and with a liquid oil, in a ratio of 1:50:667:200, respectively
(b) adding to the above mixture acetone in a ratio of 1:14;
(c) add water to the mixture in step (b) in a ratio of 1:36; and
(d) remove ethanol and acetone to obtain a nanoemulsion.

When wanting to obtain a cationic nanocapsule with astaxanthin, the method includes adding chitosan to the water of the corresponding step to form a cationic polymer solution at 0.05% w/v and then proceeding with the elimination of the solvents, or alternatively a 0.2% w/v chitosan solution is mixed with the nanoemulsion obtained. Likewise, an anionic nanocapsule with astaxanthin can be obtained by coating the cationic nanocapsule with a solution of iota carrageenan at a concentration of 0.153% w/v in a 1:1 ratio

The invention also refers to carotenoid nanostructures obtained by the proposed inventive method. In the case of a nanostructure, such nanostructure comprises carotenoids between 0.0001% w/v and 0.5% w/v; an anionic surfactant between 0.03% w/v and 3% w/v; and an oil between 0.1% w/v and 15% w/v. If it is a cationic nanocapsule, it comprises carotenoids between 0.0001% w/v and 0.5% w/v; an anionic surfactant between 0.03% w/v and 3% w/v; an oil between 0.1% w/v and 15% w/v; and a cationic polymer between 0.04% w/v and 20% w/v. If it is an anionic nanocapsule, it comprises carotenoids between 0.0001% w/v and 0.5% w/v; anionic surfactant between 0.03% w/v and 3% w/v; oil between 0.1% w/v and 15% w/v; cationic polymer between 0.04% w/v and 20% w/v; and anionic polymer between 0.00765% w/v and 0.38% w/v.

In a preferred mode of the invention, the carotenoid nanostructure is a curcumin nanostructure comprising curcumin between 0.06% and 0.07% w/v, 0.6% w/v of anionic lecithin extract and 2.36% w/v of oil. In another modality of the invention the nanostructure is a cationic nanocapsule with curcumin, comprising curcumin between 0.06% w/v to 0.07% w/v, 0.6% w/v of anionic extract of lecithin, 2.36% w/v of oil, and 4% w/v of cationic polymethacrylate. In another preferred method of the invention the nanostructure is a curcumin-containing anionic nanocapsule comprising 0.06-0.07% w/v curcumin, 0.6% w/v lecithin anionic extract, 2.36% w/v oil, 0.024% w/v polymethacrylate cation and 0.03825% w/v iota carrageenan.

Alternatively, the nanostructure in the form of a cationic nanocapsule with curcumin comprises curcumin between 0.06% w/v and 0.07% w/v, 0.6% w/v of anionic lecithin extract, 2.36% w/v of oil and 0.2% w/v of chitosan.

In another preferred form of the invention the nanostructure of the invention is a nanostructure with astaxanthin comprising 0.006% w/v astaxanthin, 0.3% w/v anionic extract of lecithin and 1.18% w/v oil. The astaxanthin nanostructure of the invention may be in the form of a cationic nanocapsule with astaxanthin comprising 0.006% w/v astaxanthin, 0.3% w/v anionic extract of lecithin, 1.18% w/v oil and 0.1% w/v chitosan or in the form of an anionic nanocapsule comprising 0.003% w/v astaxanthin, 0.15% w/v lecithin anionic extract, 0.59% w/v oil, 0.05% w/v chitosan and 0.0765% iota carrageenan.

### BRIEF DESCRIPTION OF THE FIGURES

FIG. 1 shows a photograph of 4 vials with different formulations loaded with curcumin: (a) nanoemulsions, (b) nanoemulsions coated with a layer of cationic copolymer based on dimethylaminoethyl methacrylate, butyl methacrylate and methyl methacrylate, (c) nanoemulsions coated with chitosan, (d) nanoemulsions coated with a layer of cationic copolymer based on dimethylaminoethyl methacrylate, butyl methacrylate and methyl methacrylate, and an additional layer of iota carrageenan.

FIG. 2 shows a graph of the degradation of curcumin mediated by photolysis in an oil matrix and in various formulations of nanoemulsions and nanocapsules.

FIG. 3 is a graph of curcumin degradation mediated by photolysis and oxidation (hydroxyl radical) in various formulations of nanoemulsions and nanocapsules.

FIG. 4 shows a graph of astaxanthin degradation mediated by photolysis in acetone (♦), in nanoemulsions ( [symbol] ), in chitosan nanocapsules ( [symbol] ), and in carrageenan-coated chitosan nanocapsules (●) when subjected to a photolytic stimulus.

FIG. 5 shows two graphs related to the stability of the formulations before and after being converted into a dry powder and reconstituted in water.

FIG. 6 shows images of photodegradation over time of astaxanthin in spherical hydrogels.

FIG. 7 shows images of microgels suspended in water containing nanoemulsions with astaxanthin. (A) images obtained by optical microscope, (B) by eye, and (C) transformed into a dry powder by lyophilization.

### DETAILED DESCRIPTION OF THE INVENTION

The present invention refers to a method that allows producing carotenoid nanostructures in a much simpler way than described in the state of art, without high energy requirements (since it is performed at room temperature and without complex production equipment), and with a high carotenoid encapsulation efficiency that allows reducing the loss of inputs. Also, the present invention refers to different types of nanostructures obtained with the described method, allowing to adequately dispersing the carotenoid molecules in water and providing different degrees of protection against photolysis and oxidation. It is important to note that the nanostructures can also be transformed into a dry reconstitutable powder, which gives them versatility as they remain stable for a very long time and can be dispersed in an aqueous medium that the user deems appropriate (cosmetic lotions, pharmaceuticals, soft drinks, isotonic drinks, milkshakes, soups, yoghurts, etc.) or used as an industrial input to enrich other food formulations. On the other hand, these nanostructures containing carotenoids can also be included in hydrogels of millimetric and micrometric size. This can provide systems with greater potential as it allows: i) modification of the stability and release profile of carotenoids, ii) favoring consumer acceptance due to the palatability, consistency and appearance characteristics of spherical hydrogels, and iii) favoring consumer acceptance due to the characteristics of the polymeric components that form the hydrogels (e.g. mucoadhesiveness that increases the satiety effect “decreasing hunger”).

All technical and scientific terms used here have the same meaning as understood by any person with knowledge of the state of the art where the invention belongs. However, for a better understanding of the present invention and its scope, certain technical terms used in the description of the invention will be detailed below.

In the context of the present invention, “nanostructures” shall mean a formulation with a particle size less than or equal to 500 nm, with the ability to transport, solubilize and protect from the environment active hydrophobic compounds. Such nanostructures comprise nanoemulsions and nanocapsules.

The term “nanoemulsion” refers to a mixture of two or more normally immiscible lipid and aqueous compounds, forming droplets of a size less than or equal to 500 nm and which provide surface stability by means of a surfactant.

The term “nanocapsule” refers to a nanoemulsion coated with ionic (cationic and/or anionic) polymers, which can be synthetic, semi-synthetic or natural polymers. Such nanocapsules are 500 nm or less in size and a cationic nanocapsule is designated as a nanocapsule whose outermost polymer coating is positively charged and an anionic nanocapsule as a nanocapsule whose outermost polymer coating is negatively charged.

The term “surfactant” refers to an amphiphilic molecule that can be natural or synthetic, allowing to achieve or maintain an emulsion, and which can be ionic (anionic, cationic or amphoteric) or non-ionic.

The term “organic solvent” refers to volatile organic solutions that contain carbon and are easily converted into vapors or gases and are used to dissolve raw materials, used as part of the process to form an emulsion.

The term “oil” refers to a fatty substance of mineral, vegetable or animal origin, liquid, insoluble in water, combustible and generally less dense than water, constituted by esters of fatty acids or by hydrocarbons derived from oil.

The term “polymethacrylate” refers to a cationic copolymer based on dimethylaminoethyl methacrylate, butyl methacrylate, and methyl methacrylate.

A first object of the present invention refers to a method for the production of carotenoid structures which involves mixing a carotenoid compound with an anionic surfactant, with a water-miscible organic solvent, and with a liquid oil in a particular proportion, and then pouring that mixture into an aqueous solution and shaking, and removing the organic solvent to obtain a carotenoid nanoemulsion.

In this method, either naturally or synthetically produced carotenoids can be used. For example, the nanoemulsion may contain any or a mixture of more than 700 known carotenoids, such as β-carotene, lutein, lycopene, zeaxanthin, astaxanthin, capsanthin, β-cryptoxanthin, curcumin or its derivatives (such as demethoxycurcumin, bisdemethoxycurcum in, tetrahydroxycurcum in, bis-O-demethylcurcum in (BDMC)), alloxanthin, canthaxanthin, fucozanthin, β-Apo-2′-carotenal, among others. Preferably, the method of the present invention utilizes the molecules of astaxanthin and curcumin.

The first step of the method described here involves mixing the carotenoid with an anionic surfactant with a water-miscible organic solvent, and with a liquid oil in a mass ratio of 1:5-70:10-1000:30-250, respectively. The order in which the components are mixed is irrelevant for the result expected.

Preferably, the anionic surfactant used is a lecithin anionic extract, but any anionic surfactant accepted for pharmaceutical, cosmetology, or food use may be used, such as phosphatidyl glycerol, phosphatidylserine, phosphatidylinositol, phosphatidic acid, phosphatidylcholine, phosphatidylethanolamine, and others, without limitation to the examples herein. In turn, the organic solvent that can be dissolved in water is preferably ethanol, but any organic solvent accepted for pharmaceutical, cosmetology or food use, whether of natural or synthetic origin, for example acetone, can be used, but it is not limited to these mentioned solvents. On the other hand, any type of liquid oil can be used, for example those obtained from natural sources such as coconut oil or palm oil. Any liquid oil accepted for pharmaceutical, cosmetological or food use can be used. For example, it is possible to use commercially available oils such as, but not limited to, M-5, Miglyol® 808, Miglyol® 810, Miglyol® 812, Miglyol® 818, Miglyol® 829, Miglyol® 8108, Miglyol® 840, Miglyol® 8810, Miglyol® 285 and Dynacet® 285, among others, whose high polarity makes them more solvent with active substances.

Water is added to the mixture described above in a ratio of 1:1-100, respectively. The water is preferably ultrapure by means of Milli® Q purification systems. Finally, the organic solvent is removed to obtain a nanoemulsion. The removal of the organic solvent can be done by any technique known in the state of the art for its removal. Thus, in a preferred mode of the invention, the organic solvents are removed by evaporation by means of a rotavapor.

It should be noted that the present method is performed at room temperature throughout the procedure and therefore does not require any external energy source to raise or lower the temperature. At the same time, it does not require any control of the pH of the medium to obtain the desired nanostructures.

Optionally, a second water-miscible organic solvent can be added before adding water to the mixture. This second organic solvent is preferably not the same as the first organic solvent, but the same one can be used without restriction. In a preferred form, this second solvent is acetone and is added to the mixture in a mass ratio of 1:10-20, but any other organic solvent accepted for pharmaceutical, cosmetology or food use may be used. This newly formed mixture is poured over a range of 1:1-100 of water, which is preferably ultra-pure water (distilled water purified by Milli-Q® systems) and is agitated to form a milky looking suspension. This agitation can be done manually, or magnetically, or by any agitation technique known in the state of the art. Finally, all organic solvents are removed by any technique known in the state of the art, preferably by means of rotavapor, to form the nanoemulsion.

Optionally, nanoemulsions coated with one or more layers of ionic polymers can be obtained. In a preferred mode of the invention, the method to obtain coated nano-emulsions such as a cationic nanocapsule with carotenoids, involves adding a cationic polymer to the water from the previous mentioned step to form a cationic polymer solution and then proceeding with the step to remove the organic solvent(s). Alternatively, coated nanoemulsions such as a cationic nanocapsule with carotenoids can also be obtained by adding the cationic polymer solution after the organic solvent(s) removal step. Either of these two alternatives can be developed to generate the cationic carotenoid nanocapsules.

Preferably, the cationic polymer solution is at a concentration between 0.01-2% w/v in the final mixture. This polymer solution contains a cationic polymer which can be natural, synthetic or semi-synthetic such as cationic cellulose derivatives, cationic starches, co-polymers of acrylamide salts, vinylpyrrolidone/vinylimidazole polymers, condensation products of polyglycols and amines, any of the polymers called polyquaternium, polyethyleneamine, cationic silicone polymers, dimethylamine hydroxypropyl diethylenetriamine co-polymers, cationic chitin derivatives such as quitosan and its derivatives, cationic guar gum derivatives such as guarhydroxypropyltrimonium, selected cationic gelatin proteins, gum Arabic, polyam ides, polycyanoacrylates, polylactides, polyglycolides, polyaniline, polypyrrole, polyvinylpyrrolidone, polymers of amino silicones, co-polymers of methyl methacrylate, dimethylamino methacrylate, cationic polyacrylates and polymethacrylates, among others, or any mixture thereof. In a preferred mode of the invention, a polymeric solution selected from that consisting of chitosan, cationic polymers or co-polymers based on dimethylaminoethyl methacrylate, butyl methacrylate and methyl methacrylate (whose trade name is Eudragit® E PO) is used. The polymer solution comprises the cationic polymer in an aqueous solution of ultrapure water and glacial acetic acid.

Optionally, the method of the present invention comprises adding a second coating, but this time with an anionic polymer solution bound by charges to the first cationic polymer coating, and that is stirred, thus forming the anionic nanostructures.

Preferably, the anionic polymer solution is at a concentration between 0.01-2% w/v in the final mixture. This polymer solution contains an anionic polymer that can be natural, synthetic or semi-synthetic such as carrageenan or its derivatives, carboxymethyl cellulose, alginic acid, cellulose acetate phthalate, methacrylic acid anionic co-polymers, cellulose acetate succinate, polyvinyl acetate phthalate, hydroxypropyl methyl cellulose phthalate, among others. Preferably, the polymer used is selected from the group consisting of any of the carrageenan variants, like iota carrageenan, kappa carrageenan, lambda carrageenan, etc. The polymer solution comprises the anionic polymer in an aqueous solution of ultrapure water.

In another preferred mode of the method of the present invention, the concentrations and proportions required among the previously mentioned components to obtain curcumin nanostructures in particular are specified. For this purpose, curcumin is mixed with an anionic extract of lecithin, with ethanol, and with a liquid oil, in a mass ratio of 1:8, 6:114:34, respectively; then acetone is added to this mixture in a ratio of 1:14; then water is added to that mixture in a ratio of 1:36; and finally ethanol and acetone are removed to obtain a curcumin nanostructure. The mixing parameters and forms are the same as those used to obtain carotenoid nanoemulsions.

Optionally, a cationic nanocapsule with curcumin can be obtained, when a cationic polymer is added to the water from the previous step to form a cationic polymer solution, and then the organic solvent(s) are removed, or a cationic polymer solution is added to the nanoemulsion obtained after removing the organic solvent(s). Preferably, the cationic polymer is a cationic polymethacrylate and it is found at a concentration between 0.01 and 1% w/v. Alternatively, the cationic polymer is a chitosan and at a concentration between 0.01 and 1% w/v. Additionally, the cationic nanocapsule with curcumin can be mixed with a carrageenan solution at a concentration of 0.0765% w/v in a 1:1 ratio to form an anionic nanocapsule.

In another preferred mode of the method of the present invention, the concentrations and proportions required among the previously mentioned components to obtain particularly astaxanthin nanoemulsions are specified. For this purpose, astaxanthin is mixed with an anionic extract of lecithin, with ethanol, and with a liquid oil, in a mass ratio of 1:50:667:200, respectively; then acetone is added to the before mentioned mixture in a ratio of 1:14; then water is added to this mixture in a ratio of 1:36; and finally ethanol and acetone are removed to obtain an astaxanthin nanoemulsion. The mixing parameters and forms are the same as those used to obtain carotenoid nanoemulsions.

Optionally, a cationic nanocapsule with astaxanthin can be obtained, when adding chitosan to the water from the previous mentioned step to form a 0.05% w/v cationic polymer solution. Then the organic solvent(s) are removed, or a mixture of a 0.2% w/v chitosan solution is added to the nanoemulsion obtained after removing the organic solvent(s). Additionally, the cationic nanocapsule with astaxanthin can be mixed with a 0.153% w/v carrageenan solution in a 1:1 ratio to form an anionic nanocapsule.

A second object of the present invention is a nanostructure with carotenoids comprising a nanoemulsion or a nanocapsule with carotenoids. In the case of the nanoemulsion, this comprises carotenoids between 0.0001% w/v to 0.5% w/v, an anionic surfactant between 0.03% w/v to 3% w/v, and an oil between 0.1% w/v to 15% w/v. In the case of cationic nanocapsules, it comprises carotenoids between 0.0001% w/v and 0.5% w/v; anionic surfactant between 0.03% w/v and 3% w/v; oil between 0.1% w/v and 15% w/v; and cationic polymer between 0.04% w/v and 20% w/v. In the case of anionic nanocapsules, this comprises carotenoids between 0.0001% w/v and 0.5% w/v; anionic surfactant between 0.03% w/v and 3% w/v; oil between 0.1% w/v and 15% w/v; cationic polymer between 0.04% w/v and 20% w/v; and anionic polymer 0.00765% w/v and 0.38% w/v.

Preferably, the carotenoids present in the nanostructures are selected from curcumin and astaxanthin. These nanostructures can be nanoemulsions, cationic nanocapsules or anionic nanocapsules loaded with curcumin or astaxanthin.

In a preferred mode of the present invention, the curcum in-loaded nano-emulsion is composed by curcumin between 0.06% and 0.07% w/v, lecithin anionic extract 0.6% w/v and oil between 2.36% w/v. In another preferred form of the invention, the cationic nanocapsule with curcumin comprising curcumin between 0.06% w/v to 0.07% w/v, lecithin anionic extract 0.6% w/v, oil 2.36% w/v, and a cationic polymethacrylate 4% w/v; or with curcumin between 0.06% w/v and 0.07% w/v, lecithin anionic extract 0.6% w/v, oil 2.36% w/v and chitosan 0.2% w/v. Also, the curcumin anionic nanocapsule is composed by curcumin between 0.06% and 0.07% w/v, lecithin anionic extract 0.6% w/v, oil 2.36% w/v, cationic polymethacrylate 0.024% w/v, and carrageenan 0.03825% w/v.

In another preferred form of the present invention, nanoemulsion with astaxanthin composed by astaxanthin 0.006% w/v, anionic extract of lecithin 0.3% w/v and oil 1.18% w/v; the cationic nanocapsule with astaxanthin composed by astaxanthin 0.006% w/v, lecithin anionic extract 0.3% w/v, oil 1.18% w/v and chitosan 0.1% w/v; and the anionic nanocapsule comprises astaxanthin 0.003% w/v, lecithin anionic extract 0.15% w/v, oil 0.59% w/v, chitosan 0.05% w/v and carrageenan 0.0765%.

Once the nanoemulsions and nanocapsules are formulated, they can be stored in the form of dry powder, using techniques known in the state of the art such as spray drying or lyophilization, and then reconstituted in water without losing any of the beneficial characteristics already mentioned of these nanoformulations.

All materials, methods and examples used herein are only illustrative and should not be considered in any way to limit the scope of the present invention.

### PERFORMANCE EXAMPLES

The curcumin was bought to Sigma-Aldrich™. The polymers used to produce the nanocapsules were Eudragit® E PO (Evonik Industries™), chitosan (Sigma-Aldrich™) and iota carrageenan (Gelymar™). The oil matrix was Miglyol® 812 oil (Sasol™) and the surfactant Epikuron® 145V (Cargill™). The hydrogen peroxide 30 volumes was purchased from Merck. The solvents acetone and ethanol were HPLC grade. The double-distilled water was purified by a Milli-Q® system.

### Example 1. Formulation of Curcumin and Astaxanthin Nanostructures

### Nanoemulsions Containing Curcumin

The nanoemulsions were prepared as follows: about 3.5 mg curcumin were weighed into a test tube along with 30 mg Epikuron® 145 V, then 500 μL ethanol was added and stirred in a vortex into solution. Then 125 μL of Miglyol® 812 was added, shaken and 9.5 mL of acetone was added from another test tube. The mixture was rapidly poured over 20 mL of Milli-Q® water and subjected to magnetic agitation for 5 minutes, forming a milky suspension. Finally, the solvent was evaporated to a final volume of 5 mL.

### Eudragit® E PO Cationic Nanocapsules Containing Curcumin

The cationic nanocapsules of Eudragit® E PO were prepared as follows: the same procedure used for the nanoemulsions of Example 1 was followed, but this time, after adding the 9.5 mL of acetone, the mixture was poured over 20 mL of a 1% solution of Eudragit® E PO. This solution was prepared with 1 g of Eudragit® E PO dissolved in a final volume of 100 mL Milli-Q® water, previously adding 1 mL of glacial acetic acid. The mixture was stirred for 5 minutes and then the solvent was evaporated to a final volume of 5 mL.

### Cationic Chitosan Nanocapsules Containing Curcumin

The cationic nanocapsules of chitosan were prepared in the following way: the same procedure used for curcumin-containing nanoemulsions was followed. However, this time after adding the 9.5 mL of acetone, the mixture was poured over 20 mL of a 0.05% chitosan solution. This solution was prepared with 10 mg of chitosan dissolved in a final volume of 20 mL in Milli-Q® water, after the addition of 200 μL of glacial acetic acid. The mixture was stirred for 5 minutes and then the solvent was evaporated to a final volume of 5 mL.

Cationic Nanocapsules Coated with the Anionic Polymer Carrageenan Containing Curcumin

A protocol for manufacturing anionic nanocapsules was developed by coating the cationic nanocapsules of Eudragit® E PO using a negatively charged polymer such as iota carrageenan. The same procedure used for the curcumin-containing nanocapsules was followed. However, this time after adding the 9.5 mL of acetone, the mixture was poured over 20 mL of a 0.01% solution of Eudragit® E PO. This solution was prepared with 0.002 gr of Eudragit® E PO dissolved in a final volume of 20 mL with Milli-Q® water, previously adding 0.04 mL of glacial acetic acid. The mixture was stirred for 5 minutes and then the solvent was evaporated to a final volume of 5 mL. Then, 2.5 mL of these Eudragit®E PO coated nanocapsules were mixed with 2.5 mL of 0.0765% w/v carrageenan solution and shaken for 10 min until the anionic nanocapsules were obtained.

### Nanoemulsions Containing Astaxanthin

The preparation of the nanoemulsions was the following: 0.597 mg of astaxanthin were weighed into a test tube together with 30 mg of Epikuron® 145 V, then 500 μL of ethanol were added and stirred into a vortex until dissolved. Then 125 μL of Miglyol® 812 was added, shaken and 10 mL of acetone was added from another test tube. The mixture was rapidly poured over 20 mL of Milli-Q® water and subjected to magnetic agitation for 5 minutes, forming a milky suspension. Finally, the solvent was evaporated to a final volume of 10 mL.

### Chitosan Cationic Nanocapsules Containing Astaxanthin

The cationic nanocapsules of chitosan were prepared as follows: the same procedure used for the nanoemulsions containing astaxanthin was followed, but this time, after adding the 10 mL of acetone, the mixture was poured over 20 mL of a 0.05% chitosan solution. This solution was prepared with 10 mg of chitosan dissolved in a final volume of 20 mL of Milli-Q® water, after adding 2 mL of 0.1% v/v glacial acetic acid. The mixture was stirred for 5 minutes and then the solvent was evaporated to a final volume of 5 mL.

Cationic Nanocapsules Coated with the Anionic Polymer Carrageenan Containing Astaxanthin

A protocol to produce anionic nanocapsules was developed by coating the cationic nanocapsules with chitosan using a negatively charged polymer like iota carrageenan. The same procedure used for astaxanthin-containing nanoemulsions was followed. However, this time after adding the 10 mL of acetone, the mixture was poured over 20 mL of a 0.05% chitosan solution. This solution was prepared with 10 mg of chitosan dissolved in a final volume of 20 mL in Milli-Q® water, previously adding 2 mL of glacial acetic acid at 0.1% v/v. The mixture was stirred for 5 minutes and then the solvent was evaporated to a final volume of 10 mL. Then, 4 mL of these chitosan-coated nanocapsules were mixed with 4 mL of 0.153% w/v carrageenan solution and shaken for 10 min until the anionic nanocapsules were obtained.

### Example 2. Characterization of the Formulations Developed

The final concentrations of the components in the previously described formulations were:

Nanoemulsions containing curcumin

| | | |
|---|---|---|
| Curcumin | 0.06-0.07% | p/v |
| Miglyol ® 812 | 2.36% | p/v |
| Epikuron ® 145 V | 0.6% | p/v |

Eudragit® E P O cationic nanocapsules containing curcumin

| | | |
|---|---|---|
| Curcumin | 0.06-0.07% | p/v |
| Miglyol ® 812 | 2.36% | p/v |
| Epikuron ® 145 V | 0.6% | p/v |
| Eudragit ® E PO | 4% | p/v |

Cationic chitosan nanocapsules containing curcumin

| | | |
|---|---|---|
| Curcumin | 0.06-0.07% | p/v |
| Miglyol ® 812 | 2.36% | p/v |
| Epikuron ® 145 V | 0.6% | p/v |
| Chitosan | 0.2% | p/v |

Cationic Nanocapsules Coated with the Anionic Polymer Carrageenan Containing Curcumin

| | | |
|---|---|---|
| Curcumin | 0.06-0.07% | p/v |
| Miglyol ® 812 | 2.36% | p/v |
| Epikuron ® 145 V | 0.6% | p/v |
| Eudragit ® E PO | 0.02% | p/v |
| Carrageenan | 0.03825% | p/v |

### Nanoemulsions Containing Astaxanthin

| | | |
|---|---|---|
| Astaxanthin | 0.00597% | p/v |
| Miglyol ® 812 | 1.18% | p/v |
| Epikuron ® 145 V | 0.3% | p/v |

### Chitosan Cationic Nano-Capsules Containing Astaxanthin

| | | |
|---|---|---|
| Astaxanthin | 0.00597% | p/v |
| Miglyol ® 812 | 1.18% | p/v |
| Epikuron ® 145 V | 0.3% | p/v |
| Chitosan | 0.1% | p/v |

Cationic Nanocapsules Coated with the Anionic Polymer Carrageenan Containing Astaxanthin

| | | |
|---|---|---|
| Astaxanthin | 0.002985% | p/v |
| Miglyol ® 812 | 0.59% | p/v |
| Epikuron ® 145 V | 0.15% | p/v |
| Chitosan | 0.05% | p/v |
| Carrageenan | 0.0765% | p/v |

All the formulations developed were characterized in terms of size, polydispersion index (PDI) and zeta potential using the Zetasizer Nano ZS equipment. Size of the nanoemulsion obtained: 150-250 nm. Size of the nanocapsules obtained: 150-500 nm.

The efficiency of curcumin encapsulation in the nanoformulations (referred to the percentage of curcumin that is in the nanosystems compared to that in the external aqueous phase) and the process performance (referred to the total amount of curcumin that is in the formulation (in the nanosystems and in the external aqueous phase) and compared to the amount initially added), were evaluated using conventional methods described in the literature. Table 1 shows that the encapsulation efficiency of the curcumin in the formulations is more than 90% in most cases, indicating that there is very little loss of raw material using the method proposed in the present invention. The same data was observed for formulations containing astaxanthin.

TABLE 1
Curcumin association efficiency in nanoemulsions
and different nanocapsules.

| | Curcumin at the | Curcumin in the | Association |
|---|---|---|---|
| Formulation | beginning (mg) | formulation (mg) | Efficiency (%) |
| Nanoemulsion | 3.26 | 3.21 | 98.5 |
| Eudragit E PO | 3.24 | 3.03 | 93.6 |
| Nanocapsule | | | |
| Chitosan | 3.19 | 2.91 | 91.1 |
| Nanocapsule | | | |
| Eudragit E | 3.11 | 2.80 | 89.9 |
| PO-carrageenan | | | |
| Nanocapsule | | | |

### Example 3. Comparison of the Stability of Curcumin, Subjected to Degradation Stimuli Such as Photolysis and Oxidation

### Effect of Photolysis on Different Curcumin Formulations

Between 3.2 and 3.5 mg of curcumin were dissolved in 5 mL of Miglyol® 812, or a similar amount of curcumin was studied that was contained in 5 mL of the nanoformulations (nanoemulsions, Eudragit® E PO nanocapsules, chitosan nanocapsules and Eudragit® E PO nanocapsules coated with the anionic carrageenan polymer iota); then 1.5 mL of this suspension was taken and subjected to photolysis. This volume (contained in a quartz cell) was exposed to a mercury lamp emitting a beam of light at 254 nm and 10 cm distance. The cuvette was placed in a thermoregulation device that allows the light beam to pass through a specific area at a fixed temperature of 30 degrees Celsius.

Effect of Photolysis and Oxidation (Hydroxyl Radical) in Different Formulations with Curcumin

Between 3.2 and 3.5 mg of curcumin were encapsulated in 5 mL of the different nanoformulations (nanoemulsions, Eudragit E PO nanocapsules, chitosan nanocapsules and Eudragit E PO nanocapsules coated with the anionic carrageenan polymer iota). Then 1.5 mL of these suspensions were taken, mixed with 263 μL of H₂O₂ (30% v/v), and subjected to photolysis while promoting the generation of the —OH radical (254 nm mercury lamp, 10 cm away) which is the one that generates the oxidation. The cuvette was placed in a thermoregulation device that allowed the light beam to pass through a specific area at a fixed temperature of 30 degrees Celsius.

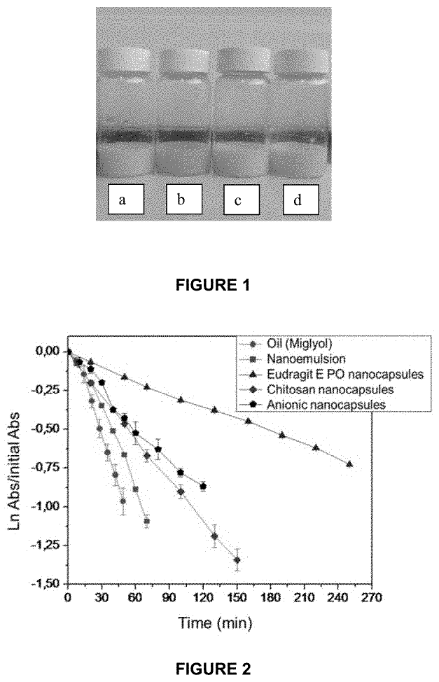

As shown in FIG. 1, (a) nanoemulsions, (b) Eudragit E PO nanocapsules, (c) chitosan nanocapsules and (d) Eudragit E PO/carrageenan nanocapsules, the nanoencapsulation strategy in various oil core systems allows the curcumin to be adequately dispersed in water.

FIG. 2 shows a graph of curcumin degradation by photolysis in Miglyol® and in various nanoformulations (nanoemulsions, Eudragit® E PO nanocapsules, chitosan nanocapsules and Eudragit® E PO/carrageenan nanocapsules called anionic nanocapsules) when subjected to a photolytic stimulus (lamp at 254 nm, 10 cm distance and 30 degrees Celsius). The “y” axis represents the ratio of absolute change in absorbance and is expressed in terms of Ln to adjust for first order degradation kinetics (n=3±D.E). In this FIG. 2 it can be seen that nanocapsules provide curcumin with a higher degree of protection against photolysis (related to a lower degradation slope) and compared to Miglyol® oil (which is the oil component that allows the molecule to dissolve inside the nanoemulsions). In Table 2, it can be seen, quantitatively, that the decreasing order of protection of all formulations towards curcumin is nanocapsules of Eudragit® E PO>nanocapsules of Eudragit® E PO/carrageenan called anionic nanocapsules>chitosan nanocapsules>nanoemulsion>oil matrix (Miglyol®).

TABLE 2
Degradation slope of the different formulations containing
curcumin and exposed to photolysis (lamp at 254
nm, 10 cm distance and 30 degrees Celsius)

| | Degradation slope |
|---|---|
| Vehicle | (min−1) |
| Oil matrix (Miglyol ®) | 0.0206 |
| Nanoemulsions | 0.0156 |
| Eudragit ® E | 0.0028 |
| PO nanocapsules | |
| Chitosan nanocapsules | 0.009 |
| Eudragit ® E | 0.0077 |
| PO/carrageenan | |
| nanocapsules (anionic | |
| nanocapsules) | |

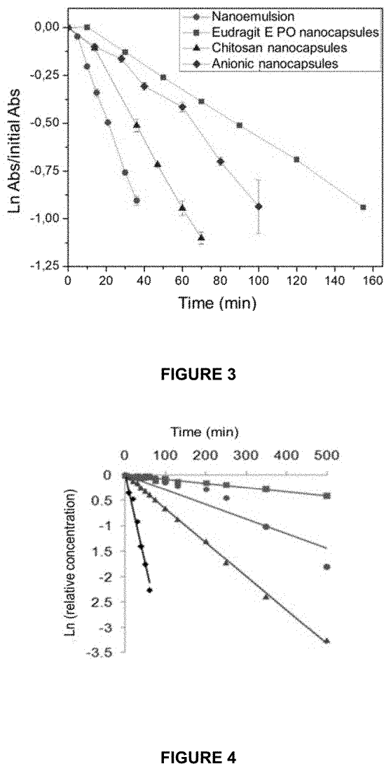

FIG. 3 shows a graph of curcumin degradation mediated by photolysis and oxidation (radical .OH) in various nanoformulations (nanoemulsions, Eudragit® E PO nanocapsules, chitosan nanocapsules and Eudragit® E PO/carrageenan nanocapsules called anionic nanocapsules) when subjected to a light stimulus (254 nm lamp, 10 cm distance and 30 degrees Celsius). The “y” axis represents the ratio of absolute change in absorbance and is expressed in terms of Ln to adjust to a first order degradation kinetic (n=3 SD). As can be seen in FIG. 3, nanoformulations provide a different degree of protection (related to a lower degradation slope) against photolysis and oxidation (mediated by the .OH radical). In Table 3, it can be seen, quantitatively, that the decreasing order of protection of all formulations towards curcumin is nanocapsules of Eudragit® E PO>nanocapsules of Eudragit® E PO/carrageenan called anionic nanocapsules>nanocapsules of chitosan>nanoemulsion. It is important to highlight that, in this case, the protection effect provided by Miglyol® oil could not be evaluated because H₂O₂ (which is the one that generates the oxidant radical .OH) is not miscible in this oil.

TABLE 3
Degradation slope of the different formulations containing
curcumin and exposed to photolysis and oxidation (•OH
radical) (lamp of 254 nm, 10 cm distance and 30 degrees Celsius).

| | Degradation slope |
|---|---|
| Vehicle | (min−1) |
| Nanoemulsion | 0.0263 |
| Eudragit ® E PO nanocapsules | 0.0062 |
| Chitosan nanocapsules | 0.0164 |
| Eudragit ® E | 0.0093 |
| PO-carrageenan | |
| nanocapsules (anionic | |
| nanocapsules) | |

Effect of Photolysis in Different Formulations with Astaxanthin

FIG. 4 shows a graph of astaxanthin degradation mediated by photolysis in acetone (♦), in nanoemulsions ( [symbol] ), in chitosan nanocapsules ( [symbol] ), and in carrageenan-coated chitosan nanocapsules ( [symbol] ) when subjected to a photolytic stimulus (254 nm lamp, 10 cm distance). The “y” axis represents the ratio of absolute change in absorbance and expressed in terms of Ln to adjust to a first order degradation kinetic (n=3±E.D.). In FIG. 4, a photolysis experiment for the carotenoid astaxanthin dissolved in the solvent acetone and in nanoemulsions and nanocapsules similar to the previous ones can be seen. The results indicate, as in the previous experiments, that it is possible to control the stability of the carotenoid by its inclusion in various nanoformulations.

### Example 4. Transformation of Nanoformulations into a Dry Powder by Lyophilization

Different concentrations of the curcum in-loaded nanoemulsions (0.5 and 1% w/v) and the presence of the cryoprotectant threalose (5 and 10% w/v) were the variables to consider transforming the nanoformulations into a dry powder and studying its reconstitution in water. The UV-Vis spectrum of curcumin from the freshly made formulations and those lyophilized and reconstituted in water were evaluated in quartz cells at a wavelength of between 350 and 550 nm. For the analysis, different aliquots (200 and 400 μL) of the water-reconstituted nano-emulsions were mixed with acetone (final volume, 5 mL) and vigorously shaken in a vortex. The formulations were then centrifuged for 30 min at 12000 G and the supernatant analyzed in the spectrophotometer.

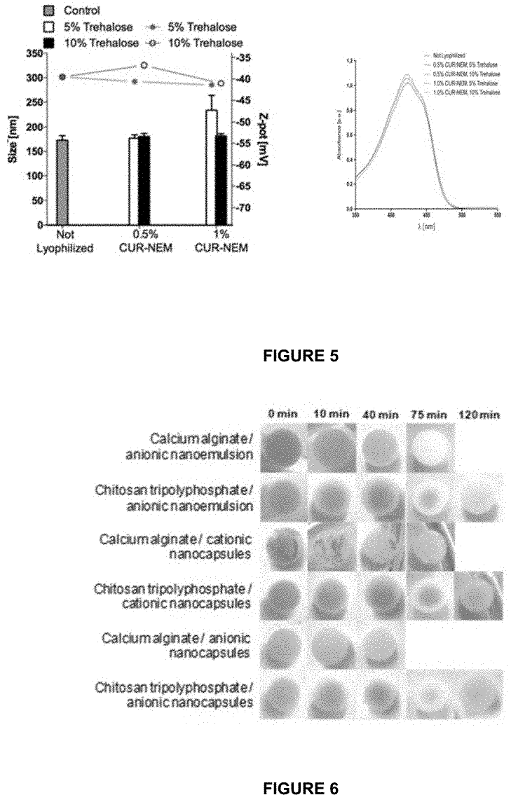

FIG. 5 shows the stability of the formulations before and after being converted into a dry powder and reconstituted in water (the effect at different concentrations of nanoemulsions and the effect of the cryoprotective agent threalose evaluated at different concentrations were analyzed). Size and zeta potential (left) and spectrum of curcumin before and after being lyophilized and reconstituted in water (right). As can be seen in FIG. 5, the nanoemulsion containing curcumin maintains its physicochemical characteristics (size, zeta potential and UV-Vis spectrum) after the suspension dispersed in water is subjected to a drying process by lyophilization and subsequent reconstitution in water.

### Example 5. Inclusion of Nanoformulations in Hydrogels

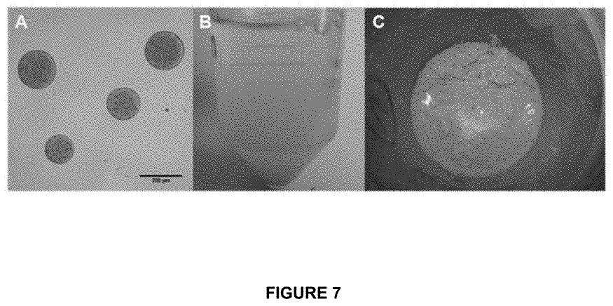

As can be seen in the following figures, it was demonstrated that the nanoformulations described here can be strategically included in spherical hydrogels of millimetric (FIG. 6) and micrometric size (FIG. 7). FIG. 6 shows the photodegradation of astaxanthin in spherical hydrogels of 2-3 millimeters diameter, and FIG. 7 shows images of calcium alginate microgels containing astaxanthin-loaded nanoemulsions suspended in water. Images obtained by (A) optical microscopy, (B) naked eye and (C) transformed into a dry powder by lyophilization can be seen. It is important to note that hydrogels containing chitosan provide greater photoprotection. Furthermore, it can be seen in image 7C that it is possible to transform these hydrogels into a dry powder.

The experimental results presented in Example 3 denote the potential of the proposed invention and describe in detail the technology used to protect and administer carotenoids orally. Although these tests are laboratory (in vitro), the technology used is simple and scalable, and considering that the systems are adequately dispersed in water, and that they can be transformed into a dry powder, these nanostructures offer a great potential for developing liquid foods or solid inputs to fortify foods and thus administer carotenoids that remain stable in the formulation and that disperse adequately in an aqueous medium.

1. A method to obtain nanostructures with carotenoids, WHEREIN said method comprises the following steps:
a) mixing a carotenoid compound with an anionic surfactant, a water miscible organic solvent and a liquid oil, in a mass ratio of 1:5-70:10-1000:30-250, respectively;
b) adding water to the mixture described in step (a) in a ratio of 1:1-100, respectively; and
c) removing the organic solvent to obtain a nanoemulsion.

2. The method according to claim 1, WHEREIN, optionally before step (b), a second water miscible organic solvent is added to the mixture in a mass ratio of 1:10-20.

3. The method according to claim 1 or claim 2, WHEREIN, in order to obtain a cationic nanocapsule, a cationic polymer is added to the water from step (b) to form a cationic polymer solution before proceeding with step (c), or a cationic polymer solution is added to the nanoemulsion obtained from step (c).

4. The method according to claim 3, WHEREIN such cationic polymer solution is at a concentration between 0.01-2% w/v in the final mixture.

5. The method according to claim 3, WHEREIN the cationic nanocapsule is optionally mixed with an anionic polymer solution in a concentration between 0.01-2% w/v in the final mixture, in order to obtain an anionic nanocapsule.

6. The method according to claim 3, WHEREIN the carotenoids are selected from curcumin and astaxanthin.

7. The method according to claim 6, WHEREIN, if such carotenoid is curcumin, said method comprises the steps of:
a) mixing curcumin with an anionic extract of lecithin, ethanol and a liquid oil, in a mass ratio of 1:8.6:114:34, respectively;
b) adding acetone to the above mixture mentioned, in a ratio of 1:14;
c) adding water to the above mentioned mixture, in a ratio of 1:36; and
d) removing ethanol and acetone to obtain a nanoemulsion.

8. The method according to claim 7, WHEREIN, in order to obtain a cationic nanocapsule with curcumin, a cationic polymer is added to the water of step (c) to form a cationic polymer solution before proceeding with step (d), or a cationic polymer solution is added to the nanoemulsion obtained from step (d).

9. The method according to claim 8, WHEREIN the cationic polymer is a cationic polymethacrylate at a concentration between 0.01 and 1% w/v.

10. The method according to claim 8, WHEREIN the cationic polymer is chitosan at a concentration between 0.01 and 1% w/v.

11. The method according to claim 8, WHEREIN in order to obtain an anionic nanocapsule, the cationic polymetacrilate-coated nanocapsule with curcumin is mixed with a solution of iota carrageenan at a concentration of 0.0765% w/v in a ratio of 1:1.

12. The method according to claim 6, WHEREIN, if such carotenoid is astaxanthin, said method comprises the steps of:
a) mixing astaxanthin with an anionic extract of lecithin, ethanol and with a liquid oil, in a ratio of 1:50:667:200, respectively;
b) adding acetone to the abovementioned mixture acetone in a ratio of 1:14;
c) adding water to the mixture in step (b) in a ratio of 1:36; and
d) removing ethanol and acetone to obtain a nanoemulsion.

13. The method according of claim 12, WHEREIN, in order to obtain a cationic nanocapsule with astaxanthin, chitosan is added to the water of step (c) to form a 0.05% w/v cationic polymer solution before proceeding with step (d), or a 0.2% w/v chitosan solution is mixed with the nanoemulsion obtained from step (d).

14. The method according to claim 13, WHEREIN, in order to obtain an anionic nanocapsule, the chitosan-coated cationic nanocapsule with astaxanthin is mixed with an iota carrageenan solution at a concentration of 0.153% w/v in a 1:1 ratio.

15. A nanostructure with carotenoids, WHEREIN said nanostructure is a nanoemulsion that comprises carotenoids between 0.0001% w/v and 0.5% w/v; an anionic surfactant between 0.03% w/v and 3% w/v; and an oil between 0.1% w/v and 15% w/v.

16. The nanostructure with carotenoids according to claim 15, WHEREIN said nanostructure is a cationic nanocapsule comprising carotenoids between 0.0001% w/v and 0.5% w/v; anionic surfactant between 0.03% w/v and 3% w/v; oil between 0.1% w/v and 15% w/v; and cationic polymer between 0.04% w/v and 20% w/v.

17. The nanostructure with carotenoids according to claim 16, WHEREIN said nanostructure is an anionic nanocapsule comprising carotenoids between 0.0001% w/v and 0.5% w/v; anionic surfactant between 0.03% w/v and 3% w/v; oil between 0.1% w/v and 15% w/v; cationic polymer between 0.04% w/v and 20% w/v; and anionic polymer 0.00765% w/v and 0.38% w/v.

18. The nanostructure with carotenoids according to claim 15, WHEREIN said carotenoids are selected from curcumin and astaxanthin.

19. The nanostructure with carotenoids according to claim 18, WHEREIN said nanostructure is a nanoemulsion with curcumin comprising curcumin between 0.06% and 0.07% w/v, 0.6% w/v of anionic extract of lecithin, and 2.36% w/v of oil.

20. The nanostructure according to claim 18, WHEREIN said nanostructure is a cationic nanocapsule with curcumin comprising curcumin between 0.06% w/v to 0.07% w/v, 0.6% w/v of anionic extract of lecithin, 2.36% w/v of oil, and 4% w/v of cationic polymethacrylate.

21. The nanostructure according to claim 18, WHEREIN said nanostructure is an anionic nanocapsule with curcumin comprising curcumin between 0.06% to 0.07% w/v, 0.6% w/v of anionic lecithin extract, 2.36% w/v of oil, 0.024% w/v of cationic polymethacrylate and 0.03825% w/v of iota carrageenan.

22. The nanostructure according to claim 18, WHEREIN said nanostructure is a cationic nanocapsule with curcumin comprising curcumin between 0.06% w/v and 0.07% w/v, 0.6% w/v of anionic extract of lecithin, 2.36% w/v of oil and 0.2% w/v of chitosan.

23. The nanostructure according to claim 18, WHEREIN said nanostructure is a nanoemulsion with astaxanthin comprising 0.006% w/v of astaxanthin, 0.3% w/v of anionic extract of lecithin, and 1.18% w/v of oil.

24. The nanostructure according to claim 18, WHEREIN said nanostructure is a cationic nanocapsule with astaxanthin comprising 0.006% w/v of astaxanthin, 0.3% w/v of anionic extract of lecithin, 1.18% w/v of oil and 0.1% w/v of chitosan.

25. The nanostructure according to claim 18, WHEREIN said nanostructure is an anionic nanocapsule comprising 0.003% w/v of astaxanthin, 0.15% w/v of anionic lecithin extract, 0.59% w/v of oil, 0.05% w/v of chitosan, and 0.0765% of iota carrageenan.
